(19) United States
(12) Patent Application Publication
(10) Pub. No.: US 2009/0175967 A1
(43) Pub. Date: Jul. 9, 2009

(54) METHODS FOR TREATMENT OF INTESTINAL CARCINOGENESIS WITH RICE BRAN

(75) Inventors: Andreas J. Gescher, Leicester (GB); Richard D. Verschoyle, Great Yarmouth (GB)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: NutraCea, Phoenix, AZ (US)

(21) Appl. No.: 12/346,736

(22) Filed: Dec. 30, 2008

Related U.S. Application Data
(60) Provisional application No. 61/019,147, filed on Jan. 4, 2008.

(51) Int. Cl. A61K 36/899; A61P 35/00

(52) U.S. Cl. 424/750

(57) ABSTRACT

The present invention provides a method for treating colorectal cancer and reducing adenoma development in mammals by administering rice bran.

### CROSS-REFERENCE TO RELATED APPLICATIONS

The present application claims priority to U.S. Provisional Patent Application No. 61/019,147, filed Jan. 4, 2007, the teaching of which is hereby incorporated by reference in its entirety for all purposes.

### BACKGROUND OF THE INVENTION

Cancers of the colorectum, breast and prostate constitute major human malignancies for which effective and safe chemoprevention strategies are scarce. Undoubtedly, several recent clinical results validate the conceptual feasibility of cancer chemoprevention in humans. These findings bear out that nonsteroidal anti-inflammatory drugs such as aspirin (Baron J A et al., N Engl J Med, 348: 891-899 (2003)) or selective inhibitors of cyclooxygenase-2 (COX-2) (Steinbach G et al., N Engl J Med, 342: 1946-1952 (2000)) interfere with colorectal malignancies, that selective estrogen receptor modulators such as tamoxifen can prevent breast cancer (Fisher B et al., J Natl Cancer Inst, 90: 1371-1388 (1998)) and that the α-reductase inhibitor finasteride can delay the onset of prostate cancer (Thompson I M et al., N Engl J Med, 349: 215-224 (2003)). However, chemopreventive interventions using drugs have raised safety concerns. For example, the recent realization that long-term administration of selective COX-2 inhibitors can detrimentally affect the cardiovascular system, resulting in an increased risk of stroke or cardiac infarction (Fitzgerald G A, N Engl J Med, 351: 1709-1711 (2004)), has dampened the enthusiasm for their extensive use as cancer chemopreventive interventions. Safety problems intrinsic to drugs administered over prolonged periods of time strengthen the interest in diet-related cancer chemoprevention approaches. Epidemiological evidence suggests that human consumption of whole-grain foods may be associated with a low incidence of cancer, especially in the colorectum (Witte J S et al., Am J Epidemiol, 144: 1015-1025 (1996); World Cancer Research Fund and American Institute for Cancer Research (1997) Patterns of diet and cancer. In Food, Nutrition and the Prevention of Cancer: a Global Perspective, Potter J D (ed) pp 20-52. Washington (DC): American Institute for Cancer Research). Rice, Oryza sativa, is the staple food of over half the world's population. The unpolished brown (bran-containing) variety possesses special dietary importance in Asia. Rice consumed in the Western world is generally white and is obtained from brown rice by removal of the bran. Dietary differences such as this may explain why the incidence of cancers, including those of the colorectum, breast and prostate is much lower in Asia than in the Western world (Witte J S et al., Am J Epidemiol, 144: 1015-1025 (1996)).

Promising cancer chemoprevention strategies can suitably be tested in genetic animal models of carcinogenesis by evaluating changes in incidence, multiplicity or volume of preneoplastic and/or neoplastic lesions. In the C3(1) SV40 T,t antigen transgenic multiple mammary adenocarcinoma (TAg) mouse, a model of breast carcinogenesis, expression of the SV40 transforming sequences (T and t antigen) is targeted to the mammary epithelium by a fragment of the rat prostatic steroid-binding protein promoter C3(1) (Maroulakou I G et al., Proc Nat Acad Sci USA, 91: 11236-11240 (1994)). The T-antigen binds and functionally inactivates p53 and Rb tumor-suppressor genes (Dyson N et al., Cell, 58: 249-255(1989); Mietz J A et al., EMBO J, 11: 5013-5020 (1992)). The consequent perturbation of cell homeostasis is thought to be responsible for mammary carcinogenesis, and all female TAg mice develop palpable tumors from approximately 12 weeks of age (Maroulakou I G et al., Proc Nat Acad Sci USA, 91: 11236-11240 (1994)). A similar genetically modified rodent species is the ‘TRansgenic Adenocarcinoma of the Mouse Prostate’ (TRAMP) model, in which expression of the SV40 transforming sequences is targeted to the prostate by a prostate-specific rat probasin promoter (Greenberg N M et al., Proc Natl Acad Sci USA, 92: 3439-3443 (1995)). All male TRAMP mice develop prostate cancer from approximately 18 weeks of age (Gingrich J R et al., Prostate Cancer Prostatic Dis, 2: 70-75 (1999)). The Apc^{Min} mouse is a model of gastrointestinal carcinogenesis genetically driven by a truncating Apc gene mutation (Luongo C et al., Cancer Res, 54: 5947-5952 (1994)), and it resembles the human heritable condition, familial adenomatous polyposis coli (FAP). Among diet-derived agents that have been found to impede carcinogenesis in these models are tea preparations, which interfered with carcinogenesis in TRAMP (Gupta S, et al., Proc Nat Acad Sci USA, 98: 10350-10355 (2001)) and TAg mice (Kaur, Greaves, Cooke, Edwards, Steward, Gescher and Marczylo, submitted), and the yellow spice curcumin, which compromised adenoma development in Apc^{Min} mice (Mahmoud N N et al., Carcinogenesis, 21: 921-927 (2000); Perkins S et al., Cancer Epidemiol Biomarkers Prev, 11: 535-540 (2002)).

In view of the foregoing, there is a need for new and effective therapies for preventing and treating carcinogenesis, for example, intestinal carcinogenesis in mammals. The present invention satisfies this and other needs.

### BRIEF SUMMARY OF THE INVENTION

As disclosed herein, the inventors show that rice products possess cancer chemopreventive properties in mammals. In one embodiment, the present invention provides a method for treating adenoma development in a mammal, the method comprising: administering rice bran to the mammal, thereby treating adenoma development. Preferably, the mammal is a human being.

In certain aspects, the rice bran is stabilized rice bran. In one aspect, the effective dose of rice bran is about 150 g to about 250 g per diem, per 70 kg body weight of the mammal.

In certain aspects, adenoma development is retarded or reduced. In certain other aspects, consumption of rice bran reduces the numbers of intestinal adenomas in the mammal. In one aspect, the rice bran exerts a physical effect, hastening fecal transit in the mammal. In another aspect, the rice bran changes the gastrointestinal microflora and luminal environment to alter the bacterial species population of the mammal. In still another aspect, the rice bran undergoes fermentation in the luminal environment, generating short chain fatty acids such as butyrate, which exert an anticarcinogenic effect.

In certain other aspects, the adenoma is an adenocarcinoma. In certain other aspects, the adenoma development such as size and/or distribution is retarded or reduced. In certain aspects, the effect of rice bran on adenoma development is more notable in small and medium-sized polyps (<3 mm), compared to larger polyps.

In still yet another embodiment, the present invention provides a method for treating colorectal cancer in a mammal, the method comprising: administering rice bran to the mammal, thereby treating colorectal cancer. Preferably, the mammal is a human being.

In certain aspects, the rice bran is stabilized rice bran.

In certain aspects, adenoma development is retarded or reduced to treat the cancer. In certain other aspects, consumption of rice bran reduces the numbers of intestinal adenomas in mammals. In one aspect, the rice bran exerts a physical effect, hastening fecal transit in the mammal. In another aspect, the rice bran changes the gastrointestinal microflora and luminal environment to alter the bacterial species population of the mammal. In still another aspect, the rice bran undergoes fermentation in the luminal environment, generating short chain fatty acids such as butyrate, which exert an anticarcinogenic effect. All of which affect cancer treatment by reducing the number or size (e.g., volume) of intestinal adenomas.

In certain aspects, the adenoma is an adenocarcinoma and development such as size and/or distribution is retarded. In certain aspects, the effect of rice bran on adenoma development is more notable in small and medium-sized polyps (<3 mm), compared to larger polyps.

In certain aspects, the effective dose of rice bran in the methods herein when calculated on the basis of equivalent body surface area, is 115 g m⁻² or 207 g per person per diem, wherein the dose assumes a body surface area of 1.8 m² accompanying a body weight of 70 kg. In other aspects, the effective dose of rice bran is about 150 g to about 250 g per diem, per 70 kg body weight. In certain instances, the effective dose is a 30% diet of rice bran. Advantageously, the results herein show that brown rice products possess cancer chemopreventive properties in mammals.

These and other aspects and embodiments will become more apparent when read with the figures and detailed description which follow.

### BRIEF DESCRIPTION OF THE DRAWINGS

FIG. 1 illustrates the effect of rice bran on whole body weight of TAg mice (A), TRAMP mice and their wild-type (C57B1/6J) counterparts (B) or Apc^{Min} mice (C). Mice received control diet or diet fortified with rice bran at 30%. Results are the mean±s.d. of 12-16 mice.

FIG. 2 illustrates the lack of effect of rice bran on mammary carcinogenesis in TAg mice as reflected by survival (A), tumor volume (B), tumor multiplicity (C) and tumor weight (D). Mice received control diet (open bars) or 30% rice bran in the diet (closed bars) from week 3 after weaning for their lifetime. Animals were sacrificed when tumor diameter exceeded 17 mm. Volume, multiplicity and weights of tumors were determined at the termination of the experiment. Results are the mean±s.d. (n=12 for controls and 15 for intervention group).

FIG. 3 illustrates the effect of rice bran on weight of the following tissues in TRAMP mice (A, C-E) or C57B1/6J (wild-type) mice (B-E): prostate carcinoma plus seminal vesicles (A), normal prostate plus seminal vesicles (B), liver (C), lung (D) and kidney (E). Mice received control diet (open bars) or diet fortified with 30% rice bran (closed bars) from weaning for their lifetime. Tissue weight was determined at the termination of the experiment. Results are the mean±s.d. of 12-16 mice. Stars indicate that liver weight in TRAMP mice was significantly lower than that in wild-type mice (*P<0.02, **P<0.005), and crosses indicate that kidney weights in mice on rice bran were significantly higher than those in mice on control diet (++P<0.002).

FIG. 4 illustrates the effect of 30% dietary rice bran on adenoma number in the small intestine (A) or colon (B) and on haematocrit (C) in Apc^{Min} mice. Mice received control diet (open bars) or diet fortified with 30% rice bran (closed bars) from week 3 after weaning for their lifetime. Results are the mean±s.d., n=15. Stars indicate that value is significantly different from control (*P<0.05, **P<0.01, ***P<0.001).

FIG. 5 illustrates the effect of dietary 30% dietary rice bran on adenoma multiplicity in the proximal (‘prox’), middle and distal sections of the small intestine (A), and on total multiplicity of small (<1 mm diameter), medium-sized (1-3 mm) or large (>3 mm) adenomas (B) in Apc^{Min} mice. Mice received control diet (open bars) or diet containing 30% rice bran (closed bars) from week 3 after weaning for their lifetime. Results, which are presented as number of adenomas per mouse as related to distribution (A) or size (B), are the mean±s.d. (n=15). Stars indicate that values are significantly different from controls (*P<0.05, **P<0.01, ***P<0.005).

FIG. 6 illustrates the lack effect of effect of 10% dietary rice bran (closed bars) or 30% dietary low-fiber rice bran (crossed bars) on adenoma number in the small intestine (A) or colon (B) and on haematocrit (C) in Apc^{Min} mice. Mice received control diet (open bars) or diet fortified with 10% rice bran (closed bars) or 30% low-fiber rice bran (crossed bars) from week 3 after weaning for their lifetime. Results are the mean±s.d., n=16-17.

### DETAILED DESCRIPTION OF THE INVENTION

### I. Embodiments

In certain aspects, the results presented herein show that rice bran (e.g., stabilized rice bran) possesses cancer chemopreventive efficacy in the Apc^{Min} mouse model of colorectal carcinogenesis. The fat content of the rice bran preparation may have caused the observed slight elevation compared to controls of TAg mouse tumor weight and volume; and of bodyweight of TRAMP, wild-type C57B16/J and female Apc^{Min} mice. The results also show that the adenoma-retarding activity of rice bran was dose-related, that it was exerted evenly along all sections of the murine intestinal tract, and that activity was associated predominantly with the fiber content of the bran rather than the nonfibrous constituents. As used herein, “an adenoma” includes for example, a collection of growths of glandular origin. Adenomas can grow from many organs including the colon, adrenal, pituitary, thyroid, etc. These growths are benign, although over time they may progress to become malignant, at which point they are called adenocarcinomas. Though adenomas are benign, they have the potential to cause serious health complications by compressing other structures (mass effect) and by producing large amounts of hormones in an unregulated, manner.

These results, obtained in three genetic carcinogenesis models, show that brown rice products possess cancer chemopreventive properties in mammals. Evidence for potential benefit seems to accumulate especially in colorectal carcinogenesis models. Rice germ (2.5% in the diet), and Kurosu, a vinegar generated from unpolished rice, prevented azoxymethane-induced colon carcinogenesis in rats (Kawabata K et al., Carcinogenesis, 20: 2109-2115 (1999); Shimoji Y et al., Nutr Cancer, 49: 170-173 (2004)). A brown rice preparation (2.5 or 5% in the diet), obtained by fermentation with Aspergillus oryzae, interfered with azoxymethane-induced formation of aberrant crypt foci and adenocarcinomas in rats (Katayama M et al., Oncol Rep, 9: 817-822 (2002)). Furthermore, fermented brown rice reduced diethylnitrosarnine- and phenobarbital-induced hepatocarcinogenesis in rats (Katayama H et al., Oncol Rep, 10: 875-880 (2003)), N-nitrosomethylbenzylamine-induced oesophageal tumorigenesis in rats (Kuno T et al., Int J Oncol, 25: 1809-1815 (2004)) and bladder carcinogenesis in mice (Kuno T et al., Oncol Rep, 15: 533-538 (2006)).

In certain aspects, the rice bran described herein is stabilized rice bran. Certain stabilized rice bran and stabilized rice bran derivatives are disclosed in the following commonly owned U.S. Patents including: U.S. Pat. No. 5,985,344, issued Nov. 16, 1999, entitled, “Process for Obtaining Micronutrient Enriched Rice Bran Oil”; U.S. Pat. No. 6,126,943, issued Oct. 3, 2000, and entitled, Method for Treating Hypercholesterolemia, Hyperlipidemia, and Atherosclerosis”; U.S. Pat. No. 6,303,586 issued Oct. 16, 2001, and entitled “Supportive Therapy for Diabetes, Hyperglycemia and Hypoglycemia”; U.S. Pat. No. 6,350,473, issued Feb. 26, 2002 and entitled “Method for Controlling Serum Glucose”; U.S. Pat. No. 6,558,714, issued May 6, 2003, and entitled “Method for Treating Hypercholesterolemia, Hyperlipidemia, and Atherosclerosis”; U.S. Pat. No. 6,733,799 issued May 11, 2004, and entitled “Method for Treating Hypercholesterolemia, Hyperlipidemia, and Atherosclerosis”; and U.S. Pat. No. 6,902,739, issued Jun. 7, 2005, and entitled “Method for Treating Joint Inflammation, Pain, and Loss of Mobility.” Each of the foregoing patents are hereby incorporated by reference.

U.S. Patent Publication No. 2007/0212470 entitled “Therapeutic uses of an anti-cancer composition derived from rice bran,” discloses methods of improving gastro-intestinal and colon health in a subject, comprising administering a rice bran compositions to a subject. The patent publication is hereby incorporated by reference. U.S. Patent Publication No. 2007/0243272 entitled “Method of treating colon cancer with rice bran composition,” and U.S. Patent Publication No. 2008/0038385, entitled “Therapeutic uses of an anti-cancer composition derived from rice bran” are also incorporated herein by reference.

Molecules in the rice bran fiber fraction, which may have contributed to inhibition of adenoma formation observed here in the Apc^{Min} mouse, comprise carbohydrates dextran-like α-glucan and arabinoxylan hemicellulose. In principle there are three types of mechanism by which dietary fiber is thought to interfere with colorectal carcinogenesis (for review, see Young G P et al., Mol Nutr Food Res, 49: 571-584 (2005)), and they may also be germane to rice bran fiber. These mechanisms have been the subject of much investigation, although details and their relative importance are unresolved. Firstly, fiber is thought to exert ‘physical’ effects such as increasing fecal bulk, hastening fecal transit and binding potentially co-carcinogenic bile salts. Secondly, fiber can change the gastrointestinal microflora and luminal environment in such a way as to alter bacterial species, that may, in turn, reduce bile salt metabolism. Thirdly, fiber carbohydrates can undergo fermentation in the luminal environment, generating short chain fatty acids such as butyrate, which are thought to exert anticarcinogenic effects such as inhibition of cell proliferation and induction of differentiation and apoptosis.

The rice bran dose in humans that would be equivalent to the 30% dietary dose (˜36 g kg⁻¹ pd), which was active in the mice described here, when calculated on the basis of equivalent body surface area, would be 115 g m⁻² or 207 g per person pd, assuming a body surface area of 1.8 m² accompanying a body weight of 70 kg (Freireich E J et al., Cancer Chemother Rep, 50: 219-244 (1966)). The dose herein is about 150 g to about 250 g pd for 70 kg of body weight. More particularly, about 175 g to about 225 g pd for 70 kg of body weight, and even more particularly, about 190 g to about 210 g pd for 70 kg of body weight.

In certain aspects, an investigation of effects of 30% dietary rice bran on the murine organism, as reflected by weights of the whole body, liver, lung, kidney or prostate, did not uncover untoward side-effects. The only unusual effect was a small increase in kidney weight associated with a slight increase in tubular lipid, content, but without associated degenerative alterations. In certain instances, it was also discovered that livers in TRAMP mice were slightly but significantly smaller than those in their wild-type counterparts, a phenomenon that has not been reported thus far. One may speculate that prostate tumors secrete agents that specifically retard liver weight gain.

A retrospective analysis of results in rodents and humans, obtained with inhibitors of COX enzymes in colorectal cancer prevention, shows reasonable consistency (Hawk E T and Levin B, J Clin Oncol, 23: 378-391 (2005)). The Apc^{Min} mouse model predicted the adenoma-regressing activity of the NSAID sulindac and the COX-2 inhibitor celecoxib (Boolbol S K et al., Cancer Res, 56: 2556-2560 (1996); Jacoby R F et al., Cancer Res, 60: 5040-5044 (2000)) in familial adenomatous polyposis coli patients (Giardiello F M et al., N Engl J Med, 328: 1313-1316 (1993); Steinbach G et al., N Engl J Med, 342: 1946-1952 (2000)). Unwanted side effects of NSAIDs and of COX-2 inhibitors, the latter of which have recently received considerable attention (Fitzgerald G A, N Engl J Med, 351: 1709-1711 (2004)), may ultimately militate against their extensive use as cancer chemopreventive agents in humans. Therefore, the search for toxicologically innocuous alternative interventions is timely and propitious, and foodstuffs provide an attractive focus for this search. In the light of the safety of rice bran (e.g., stabilized rice bran) and its efficacy in the Apc^{Min} mouse described here, rice bran is useful to prevent adenoma recurrence.

A hypothesis that rice bran interferes with development of tumors in TAg, TRansgenic Adenocarcinoma of the Mouse Prostate (TRAMP) or Apc^{Min} mice, genetic models of mammary, prostate and intestinal carcinogenesis, respectively was tested. Mice received rice bran (30%) in AIN-93G diet throughout their post-weaning lifespan. In TAg and TRAMP mice, rice bran did not affect carcinoma development. In TRAMP or wild-type C57B16/J mice, dietary rice bran increased kidney weight by 18 and 20%, respectively. Consumption of rice bran reduced numbers of intestinal adenomas in Apc^{Min} mice by 51% (P<0.01), compared to mice on control diet. In parallel, dietary rice bran decreased intestinal hemorrhage in these mice, as reflected by increased haematocrit. At 10% in the diet, rice bran did not significantly retard Apc^{Min} adenoma development. Likewise, low-fiber rice bran (30% in the diet) did not affect intestinal carcinogenesis, suggesting that the fibrous constituents of the bran mediate chemopreventive efficacy. The results indicate that rice bran is a chemopreventive intervention in humans with intestinal polyps.

### II. EXAMPLES

### Materials and Methods

### Animals

Breeding colonies were established with: (i) TAg mice on an FVB background, (ii) TRAMP mice on a C57BL/6J background and (iii) C57BL/6J Min/+(Apc^{Min}) mice. Mice were bred in the Leicester University Biomedical Services facility using animals originally obtained from either the Jackson Laboratory (Bar Harbor, Me., USA, Apc^{Min} and TAgs) or the NCI Mouse Repository (NCI Frederick Rockville, Md., USA, TRAMP). Ear tissue from newborn mice was genotyped for the presence of the transgene using PCR, as described previously (Maroulakou I G et al., Proc Nat Acad Sci USA, 91: 11236-11240 (1994); Perkins S et al., Cancer Epidemiol Biomarkers Prev, 11: 535-540 (2002); The Jackson Laboratory website: www.jax.org).

### Rice Bran

Two stabilized rice bran preparations (‘Rice X Stabilized Rice Bran—Regular’ and ‘Rice X Solubles’), produced by the Rice X Comp (El Dorado Hills, Calif., USA), were either purchased from Alexander Essentials (Morecambe, UK) or obtained as a gift from the Rice X Comp. ‘Rice X Stabilized Rice Bran—Regular’, referred to in the following as ‘rice bran’, is produced by milling brown rice, a process which releases an active lipase. The milling process includes a ‘stabilization’ step involving elevated temperature and pressure to ensure lipase deactivation. According to the providers' product data sheet, ‘Rice X Stabilized Rice Bran—Regular’ contains (all values expressed per 100 g rice bran) 29 g dietary fiber, 15 g protein, 21 g fat and 22 g available carbohydrate. This bran preparation contains the following vitamins and minerals: carotenoids (129 μg), vitamin B complex (57 mg), vitamin E complex (26 mg), folic acid (27 μg), biotin (14 μg), choline (105 mg), inositol (1496 mg) γ-oryzanol (245 mg), phytosterols (341 mg), sodium (8 mg), potassium (1573 mg), calcium (40 mg), magnesium (727 mg), phosphorus (1591 mg), manganese (26 mg), iron (8 mg) and zinc (6 mg). ‘Rice X Solubles’ (referred to in the following as ‘low-fiber rice bran’) is a powdered emulsion of soluble stabilized rice bran omitting insoluble fiber. It contains (all values per 100 g rice bran) 3 g dietary fiber, 8 g protein, 27 g fat and 55 g available carbohydrate. This bran preparation contains the following vitamins and minerals: carotenoids (47 μg), vitamin B complex (92 mg), vitamin E complex (18 mg), folic acid (37 μg), biotin (15 μg), choline (150 mg), inositol (1314 mg), γ-oryzanol (248 mg), phytosterols (413 mg), sodium (16 mg), potassium (1562 mg), calcium (8 mg), magnesium (171 mg), phosphorus (763 mg), manganese (3 mg), iron (2 mg) and zinc (2 mg).

### Animal Experiments

Experiments were carried out under animal project license PPL 40/2496, granted to Leicester University by the UK Home Office. The experimental design was vetted by the Leicester University Local Ethical Committee for Animal Experimentation and met the standards required by the UKCCCR guidelines (Workman P et al., Br J Cancer, 77: 1-10 (1998)). Groups of 10-16 mice at 4 weeks of age received standard AIN 93G diet or AIN diet supplemented with rice bran (30% or 10%) to the end of the animals' life. No attempt was made to adjust nutritional components of the AIN93 diet to compensate for the addition of rice bran. The calorific values of AIN 93 G diet or AIN diet containing 30% rice bran or 30% low-fiber rice bran were 377, 363 and 410 calories, respectively, per 100 g diet (based on Reeves P G et al., J Nutr, 123: 1939-1951 (1993), and the rice bran provider's product data sheets). Addition of rice bran decreased the protein and carbohydrate content of the overall diet slightly, and increased the fat content from 7% in control AIN diet to 11% for the diet containing 30% rice bran and to 14% for the low-fiber rice bran diet (30%). From 11 weeks of age, TAg and TRAMP mice were palpated once or twice weekly for presence of tumors. TAg tumor size was measured using callipers, and tumor volume was calculated using the equation: V=0.5236×D×d², with D and d representing the long and the short diameters, respectively. Animals were killed by cardiac exsanguination (halothane anesthesia) in weeks 18 (Apc^{min}), 19 (TAg) or 34 (TRAMP). The intestinal tract of Apc^{Min} mice was removed and flushed with phosphate-buffered saline. Intestinal tissue was cut open longitudinally and examined under a magnifying lens. Multiplicity, location and size of adenomas were recorded as described before (Perkins S et al., Cancer Epidemiol Biomarkers Prev, 11: 535-540 (2002)).

Packed red cell volume (haematocrit) was measured as described before (Strumia M M et al., Am J Clin Pathol, 24: 1016-1024 (1954)). Tumors in TAg mice, prostate with prostate tumor plus seminal vesicles, livers, kidneys and lungs of wild-type C57BL6/J or TRAMP mice were excised, weighed and placed in buffered formalin (for histopathology).

### Histopathology

The following tissues were fixed in formalin for a minimum of 2 weeks: the pelt from TAg mice, the intestinal tract from Apc^{Min} mice, the prostate with prostate tumor plus seminal vesicles and lungs, liver, salivary glands, kidney, spleen, pancreas, gut and dorsal abdominal connective tissue, including inguinal and lumbar lymph nodes from TRAMP mice or wild-type mice. TAg mouse pelts were cut to yield five transverse blocks corresponding to the five pairs of mammary glands. All tissues were embedded in paraffin wax and sections (5 μm thick) were cut and stained with haematoxylin and eosin before microscopic examination. For estimation of numbers of microadenomatous crypts in the colorectum of Apc^{Min} mice, the formalin-fixed colorectal tract was placed in 0.5% aqueous methylene blue solution (20 s). Excess stain was removed (water), and tissue was flattened between two microscope slides (held in place with elastic bands) and scanned microscopically.

### Statistical Evaluation

Evaluation of significance of values, as compared to the appropriate controls, was performed by either one-way analysis of variance with subsequent Tukey's pairwise comparison or a two-sample Student's t-test.

### Results

### Effect of Rice Bran on Murine Body Weight

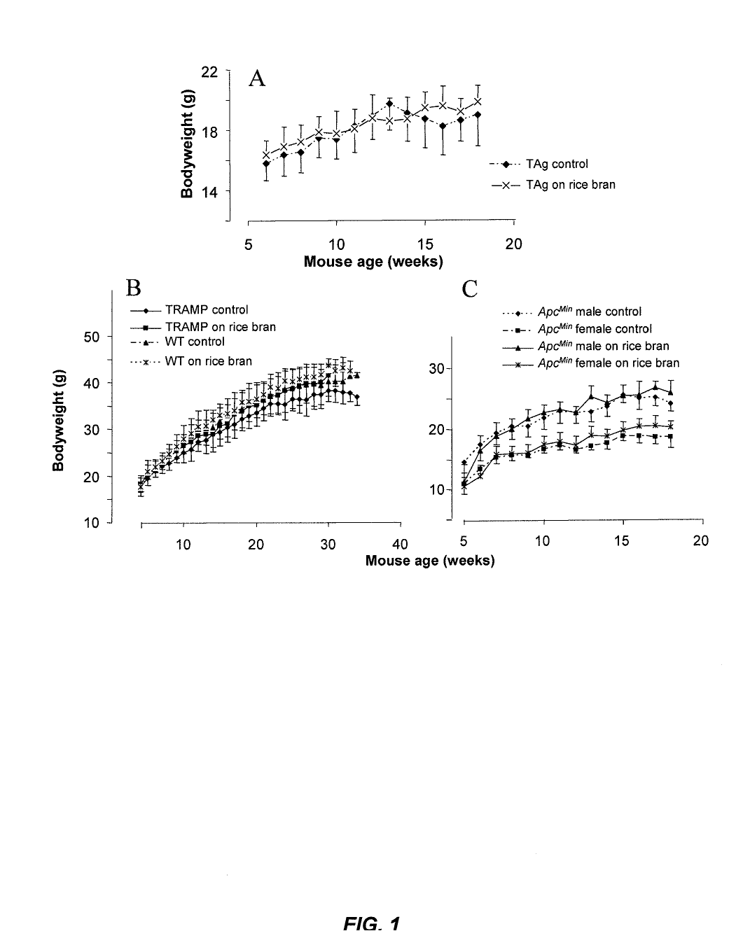

TAg, TRAMP, their C57B1/6J wild-type counterparts and Apc^{Min} mice received rice bran in their diet (30%,˜0.9 g per mouse=˜36 g kg⁻¹ per day) from weaning until the end of the experiment, which was week 18 for the Apc^{Min} mice, week 19 for the TAg mice and week 34 for the TRAMP mice. FIG. 1A shows that the animals' body weight was not significantly different from that of mice on the control diet. There is an indication that TRAMP, wild-type C57B1/6J and female Apc^{Min} mice on rice bran were marginally heavier than those on AIN diet alone (FIG. 1B, C). Overall, this result suggests that rice bran in the diet does not adversely affect food intake.

### Effect of Rice Bran on TAg and TRAMP Mice

On histopathological investigation, TAg mice presented with intra-duct hyperplasia, intra-duct carcinoma and invasive mammary carcinoma, occasional intra-duct papillomas were also present. In TRAMP mice, proliferative lesions in the prostate formed a continuum between increasing degrees of glandular hyperplasia with atypical cytological features through to frank adenocarcinoma without clearly defined nodular benign neoplasia or adenoma. These observations are consistent with the original description of the TAg and TRAMP mouse models (Maroulakou I G et al., Proc Nat Acad Sci USA, 91: 11236-11240 (1994); Gingrich J R et al., Prostate Cancer Prostatic Dis, 2: 70-75 (1999)). There was no clear difference between control and treated mice with respect to TAg or TRAMP tumor histopathology.

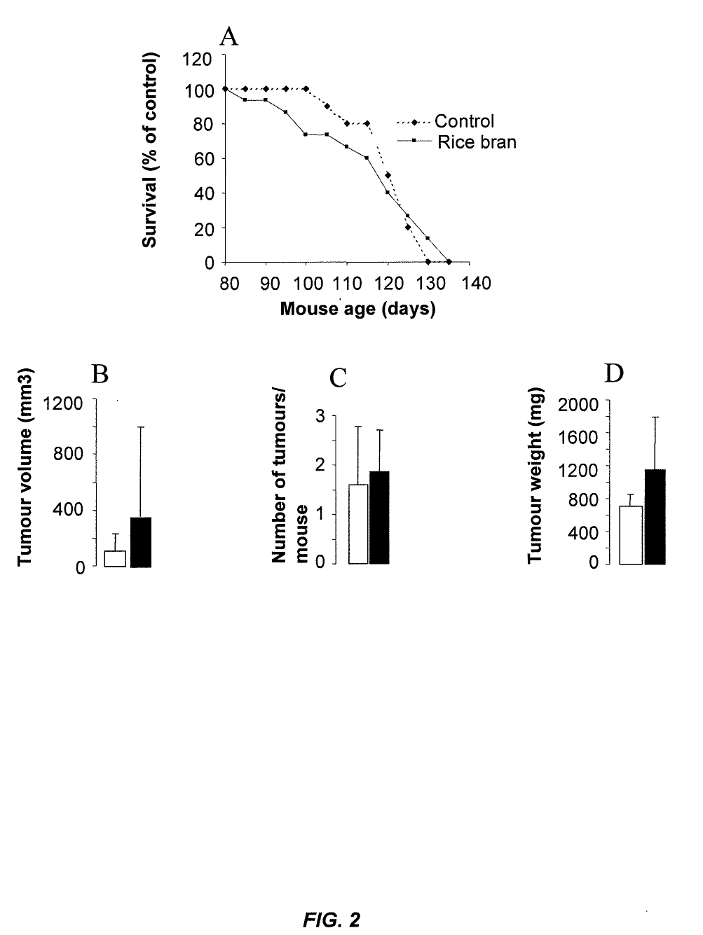

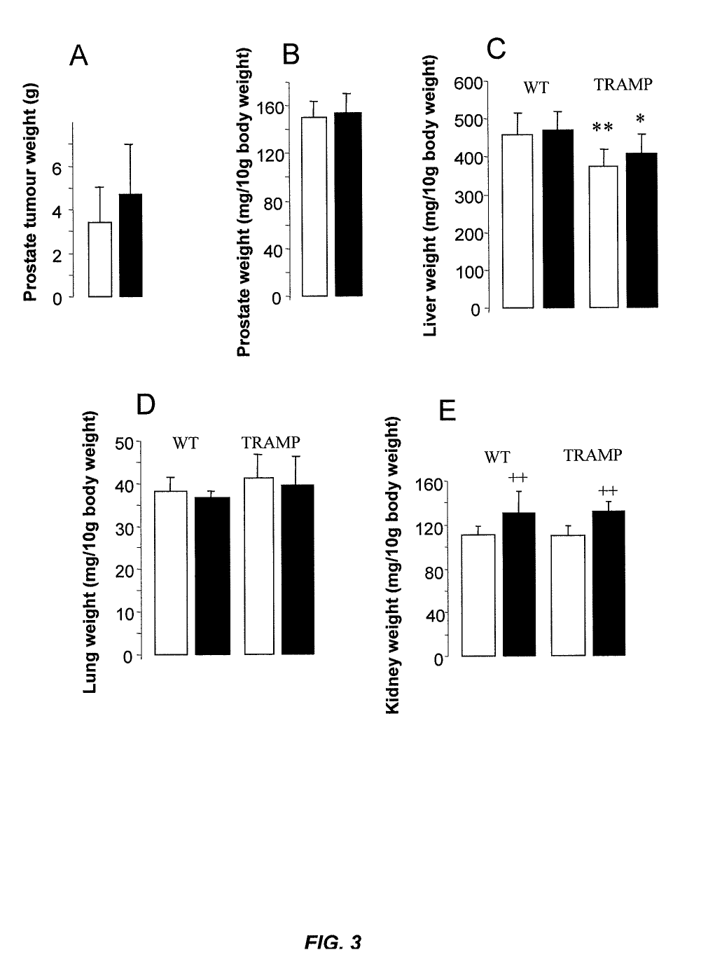

Consumption of rice bran failed to significantly affect mammary carcinogenesis in TAg mice, as reflected by survival of animals (FIG. 2A), tumor volume (FIG. 2B), number of tumors per mouse (FIG. 2C) or tumor weight at the end of the experiment (FIG. 2D). The results shown in FIG. 2B and D tentatively hint at slightly increased tumor weight and volume in TAg mice on rice bran compared to controls. Rice bran consumption did not interfere with prostate carcinogenesis in TRAMP mice, as mirrored by tumor weight at the end of the experiment (FIG. 3A).

Healthy prostate, liver, kidney, lung spleen, lymph nodes, pancreas and gut tissues in TRAMP and wild-type C57B1/6J mice were closely inspected for potential effects of rice bran. The weight of healthy prostate in C57BL/6J (FIG. 3B) and of liver and lung in wild-type C57BL/6J or TRAMP mice (FIG. 3C and D) was not affected by rice bran consumption. Livers in TRAMP mice weighed significantly less than those in wild-type mice, irrespective of diet (FIG. 3C). For mice on control diet, the difference was 18% and for mice on rice bran 13%. These livers showed variable degree of clear cell change (glycogen) and vacuolation (fat) typical of well-fed mice without clear histological differences between rice bran-fed and control mice. Kidneys in wild-type or TRAMP mice that received 30% rice bran weighed 20 or 18%, respectively, more than kidneys in mice on control diet (FIG. 3E). On histopathological inspection, large clear vacuoles, indicative of lipid droplets without evidence of cellular degeneration, were present in the proximal tubular cell cytoplasm in all mice, albeit more prominently in the mice that received rice bran.

### Effect of Rice Bran on Intestinal Adenoma Development in Apc^{Min} Mice

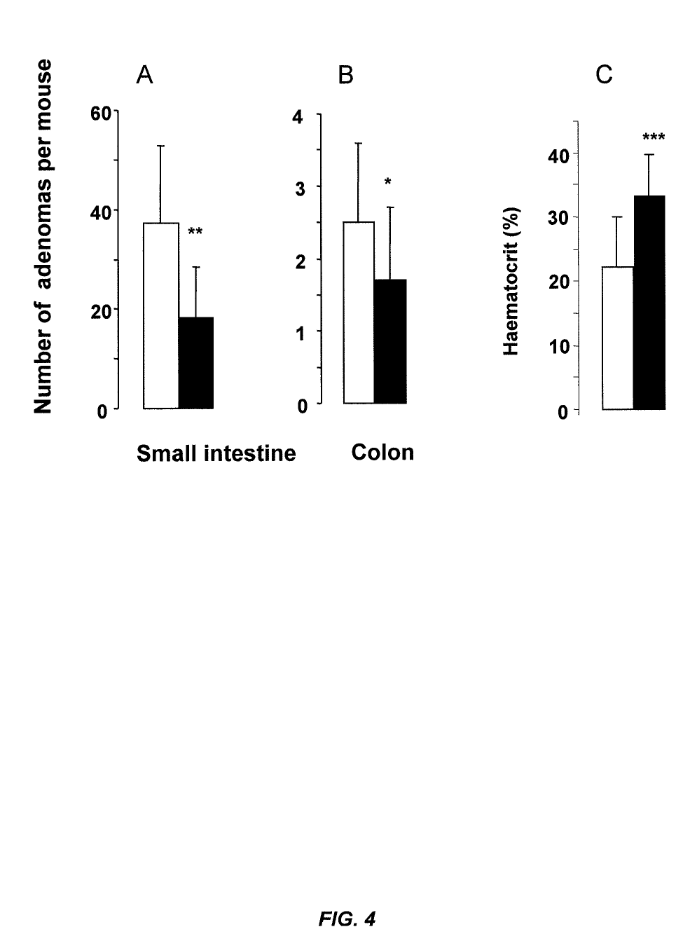

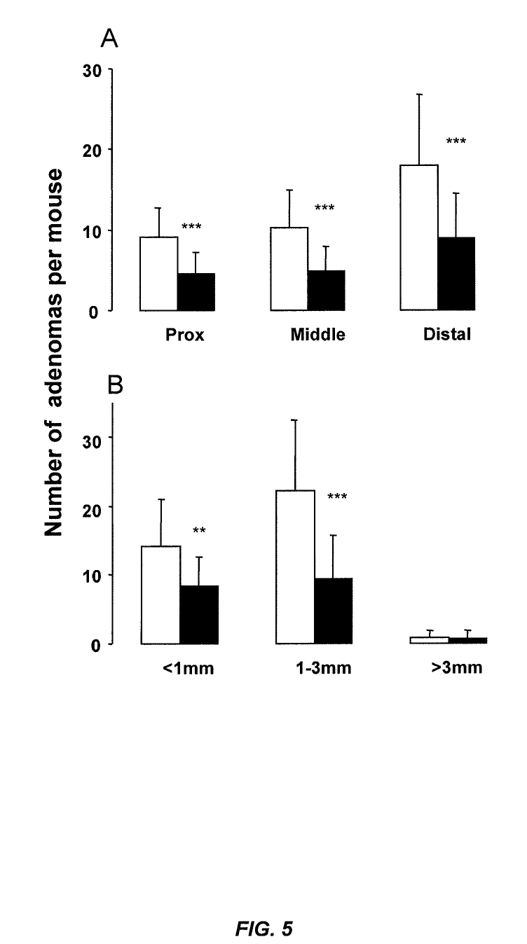

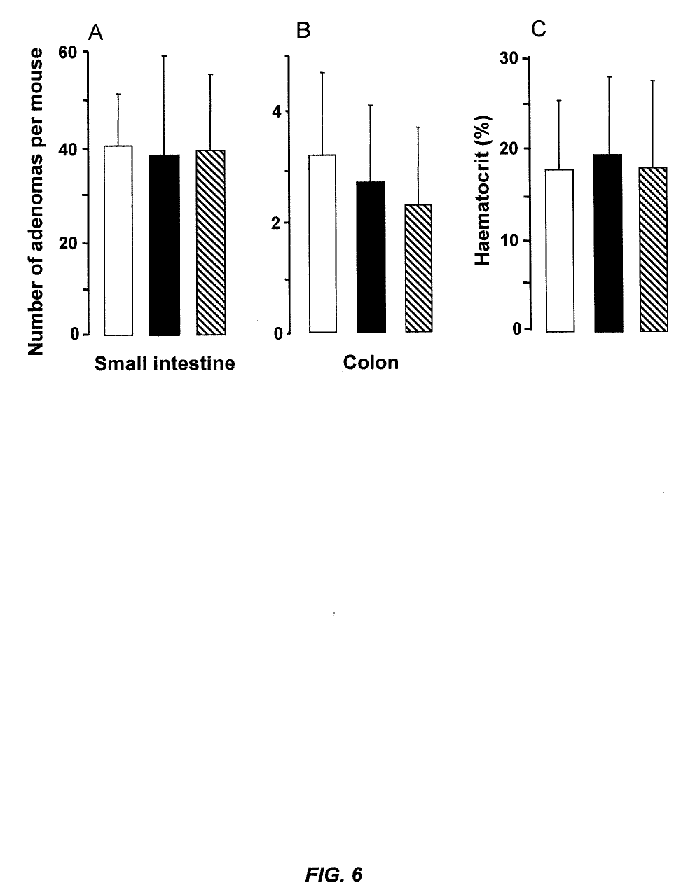

Histopathological analysis of the small intestine of Apc^{Min} mice, which had received control diet or rice bran (30%) during their life time, showed focal proliferative lesions ranging from hyperplastic glands to larger areas of glandular hyperplasia and polypoid adenomas. There were no significant differences in tumor morphology in terms of dysplasia between control mice and mice on rice bran, which suggests that there was no difference in tumor aggressiveness. The numbers of adenomas in the small intestine or colon of mice on rice bran were significantly reduced by 51 and 32%, respectively, compared to mice on control diet (FIG. 4A and B). A detailed analysis of small intestinal polyp location revealed that polyp numbers in the proximal, middle and distal sections of the intestine were similarly affected by rice bran (FIG. 5A). The effect of rice bran on adenoma development was more notable in small and medium-sized polyps (<3 mm) than in large ones (>3 mm) (FIG. 5B), even though the overall small number of large polyps observed may have obfuscated any difference. At the late stage of adenoma development, Apc^{Min} mice suffer from intestinal bleeding, which causes a dramatic fall in haematocrit. Intervention with rice bran raised the haematocrit measured at the end of the experiment, from 22.2% in untreated Apc^{Min} mice to 33.1%, consistent with impeded adenoma development (FIG. 4C). In order to test the hypothesis that a smaller dose of rice bran may affect adenoma development, the experiment was repeated by including mice on 10% dietary rice bran. In this repeat experiment, rice bran at 30% reduced small intestinal adenoma load by 45% (from 40±11 per mouse to 22±15, mean±s.d., n=15-17, P<0.005) and colonic adenomas by 31% (from 3.2±1.5 to 2.2±1.1, P<0.05), in accordance with the results shown in FIG. 4. In contrast, small intestinal or colonic adenoma numbers were not significantly diminished by 10% rice bran (FIG. 6A and B). Consistent with these results, the haematocrit in Apc^{Min} mice on 30% rice bran (28.6±9.9%) was significantly higher than that in mice on the control diet (17.7±7.6%, P=0.002), whereas 10% rice bran did not significantly increase the haematocrit compared to controls (FIG. 6C).

In order to explore whether nonfibrous-constituents of rice bran mediate the retardation of adenoma development, Apc^{Min} mice received a low-fiber rice bran preparation with their diet at the same dose (30%), at which high-fiber rice bran reduced adenomas. The numbers of adenomas in the small intestine and colon of mice on low-fiber rice bran were not significantly different from those in mice on control diet (FIG. 6A and B), and the haematocrit reflected the lack of effect of low-fiber rice bran on adenoma development (FIG. 6C). Thus, in contrast to high-fiber-containing rice bran, low-fiber rice bran failed to affect adenoma formation.

It has been suggested that numbers of microadenomatous crypts in the colorectal tract of Apc^{Min} mice might allow a judgment to be made as to the effect of potential chemopreventive interventions, in a fashion arguably more relevant to the human disease counterpart than by counting adenomas in the small intestine (Yamada Y et al., Cancer Res, 62: 6367-6370 (2002)). When we enumerated the microadenomatous crypts identifiable in the colorecta of Apc^{Min} mice in this experiment, 16 of the 51 mice (31%) had a total of 37 microadenomatous crypts. Individually, the mice displayed between one and seven lesions, far fewer than the >20 lesions per mouse observed by Yamada Y et al., Cancer Res, 62: 6367-6370 (2002). There was no difference in propensity between mice on control diet and those on rice bran to bear microadenomatous crypts.

All publications, patents and patent applications mentioned in this specification are herein incorporated by reference into the specification in their entirety for all purposes. As will be apparent to persons skilled in the art, modifications and adaptations to the above-described invention can be made without departing from the spirit and scope of the invention, which is defined and circumscribed by the appended claims.

What is claimed is:

1. A method for treating, reducing or preventing reoccurrence of adenoma development in a mammal, said method comprising:
administering rice bran to said mammal, thereby treating adenoma development.

2. The method of claim 1, wherein said mammal is a human being.

3. The method of claim 2, wherein the effective dose of rice bran is about 150 g to about 250 g pd per diem, per 70 kg body weight.

4. The method of claim 1, wherein said rice bran is stabilized rice bran.

5. The method of claim 1, wherein adenoma development is retarded or reduced.

6. The method of claim 1, wherein said rice bran exerts a physical effect, hastening fecal transit in said mammal.

7. The method of claim 1, wherein said rice bran changes the gastrointestinal microflora and luminal environment to alter the bacterial species population of said mammal.

8. The method of claim 1, wherein said rice bran undergo fermentation in the luminal environment, generating short chain fatty acids such as butyrate, which exert an anticarcinogenic effect.

9. The method of claim 1, wherein said adenoma is an adenocarcinoma.

10. The method of claim 1, wherein said adenoma size and/or distribution development is retarded.

11. A method for treating or reducing the incidence of colorectal cancer in a mammal, said method comprising:
administering rice bran to said mammal, thereby treating colorectal cancer.

12. The method of claim 11, wherein said mammal is a human being.

13. The method of claim 12, wherein the effective dose of rice bran is about 150 g to about 250 g per diem, per 70 kg body weight.

14. The method of claim 11, wherein said rice bran is stabilized rice bran.

15. The method of claim 11, wherein adenocarcinoma development is retarded or reduced.

16. The method of claim 11, wherein said rice bran exerts a physical effect, hastening fecal transit in said mammal.

17. The method of claim 11, wherein said rice bran changes the gastrointestinal microflora and luminal environment to alter the bacterial species population of said mammal.

18. The method of claim 11, wherein said rice bran undergo fermentation in the luminal environment, generating short chain fatty acids such as butyrate, which exert an anticarcinogenic effect.

19. The method of claim 11, wherein said adenoma size and/or distribution development is retarded.

20. A method of chemopreventive intervention in a human with existing intestinal polyps, said method comprising:
administrating rice bran at a dose of about 150 g to about 250 g per diem, per 70 kg body weight, thereby preventing or reducing cancer in said human with existing intestinal polyps.
